(19) United States
(12) Patent Application Publication
(10) Pub. No.: US 2025/0224407 A1
(43) Pub. Date: Jul. 10, 2025

(54) SYSTEM FOR EVALUATING OVARIAN RESERVE FUNCTION OF SUBJECT

(71) Applicants: PEKING UNI. THIRD HOSPITAL (THE THIRD CLINICAL MEDICAL SCHOOL OF PEKING UNI.), Beijing (CN); HANGZHOU QINGGUO MEDICAL TECHNOLOGY CO., LTD., Zhejiang (CN)

(72) Inventors: Huiyu XU, Beijing (CN); Rong LI, Beijing (CN); Jie QIAO, Beijing (CN); Guoshuang FENG, Zhejiang (CN); Yong HAN, Zhejiang (CN)

(73) Assignees: PEKING UNI. THIRD HOSPITAL (THE THIRD CLINICAL MEDICAL SCHOOL OF PEKING UNI.), Beijing (CN); HANGZHOU QINGGUO MEDICAL TECHNOLOGY CO., LTD., Zhejiang (CN)

(21) Appl. No.: 18/857,302

(22) PCT Filed: May 13, 2021
(86) PCT No.: PCT/CN2021/093551

§ 371 Date: Oct. 16, 2024

(30) Foreign Application Priority Data
Apr. 22, 2021 (CN) 202110438776.1

(51) Int. Cl. G01N 33/68

(52) U.S. Cl. CPC G01N 33/689; G01N 2333/575; G01N 2800/367

(57) ABSTRACT

The present invention relates to a system for optimally evaluating a reserve function and an ovarian youth degree of a subject. The system comprises: a data collection module, which is used for acquiring data of an age and an anti-Müllerian hormone (AMH) level of a subject; and a module for calculating an ovarian reserve function or an ovarian youth degree, which is used for performing calculation on the information acquired in the data collection module, so as to calculate the probability (p) of a poor ovarian response of the subject and obtain a youth degree score, and for evaluating the ovarian youth degree according to the youth degree score.

### TECHNICAL FIELD

The present invention relates to a system and method for evaluating the ovarian reserve function of a subject and the level of the ovarian youth index (referred to simply as ovarian youth index) of a subject, and the system or method can be used to evaluate the subject's own ovarian reserve function or the level of the ovarian youth index of the subject to evaluate reproductive potential thereof, and to evaluate whether the reproductive potential of the subject is improved after corresponding treatment.

### BACKGROUND

The number of primordial follicles contained within the ovarian cortex is called the ovarian reserve. It reflects the ability of the ovary to provide healthy, fertile eggs and is the most important marker for women's ovarian function. Generally speaking, the higher the number of primordial follicles, the better the quality and the higher the probability of conception.

Ovarian reserve increases and gradually depletes with age, which is the main internal cause of female aging. Therefore, evaluating ovarian reserve is also evaluating women's ovarian youth index.

With the depletion of the number of primordial follicles, women's ovarian function is close to exhaustion, entering perimenopause, and the ovarian youth index is close to 0. Decreased ovarian reserve is the main reason for the decline of female fertility. However, there are large individual differences in ovarian reserve. Currently, the evaluation of ovarian reserve mainly relies on personal experience, and there is no intelligent method for evaluating ovarian reserve, that is, ovarian youth index.

The evaluation of ovarian reserve score and ovarian youth indexovarian youth index can help women of childbearing age understand their fertility status, so as to arrange their fertility plans reasonably. For women with a history of infertility, it can be used to predict ovarian responsiveness in women of childbearing age, and provide a reference for clinical diagnosis and treatment planning of infertility. At present, the main basis for the diagnosis of decreased ovarian reserve function is the prediction of poor ovarian response in Bologna criteria. Therefore, the indicators for evaluating ovarian reserve function are actually indicators for predicting ovarian responsiveness.

Age is an important factor in evaluating ovarian reserve. A study on age and IVF success rates showed that the IVF success rate for women under the age of 30 is about 26%, while the IVF success rate is only 9% when the age is 37 years and older.

In the field of reproductive medicine, the purpose of evaluating ovarian reserve is to predict ovarian responsiveness. At present, serum AMH level is recognized as the indicator having best correlation with ovarian reserve function, because AMH has a good correlation with antral follicle count (AFC) and serum basal FSH level. It is theoretically possible to replace AFC and FSH levels with AMH through appropriate model exploration. So in the patent application, the inventors tried to build such a model.

Antral follicle count (AFC) is the number of follicles less than 8 mm in diameter in early Gn-dependent follicular growth. It is well known that the primordial follicle pool in the ovary is related to the number of growing antral follicles, so, in theory, the AFC can reflect the remaining ovarian follicle pool as accurately as possible. However, obtaining good AFC results requires ultrasonography by a skilled transvaginal sonography (TVS) specialist, which is time-consuming and resource-intensive. There is a lack of standards in AFC measurement, and AFC varies with factors such as menstrual cycle, contraceptive use, and sensitivity and resolution of TVS devices. All of these existing confounding factors can make reliable assessment of AFC more difficult.

The inventor's previous patent application CN201811516206.4 provides a system for evaluating an ovarian reserve function of a subject, comprising: a data collection module for acquiring data of the age, anti-Mullerian hormone (AMH) level, follicle-stimulating hormone (FSH) level and antral follicle count (AFC) of the subject; and a module for calculating the ovarian reserve function, which is used for calculating the information acquired in the data collection module, so as to calculate the probability (p) of poor ovarian response of the subject. In this system, the receiver operating characteristic (ROC) curve is used to detect the cut-off points of age, anti-Mullerian hormone (AMH) level, follicle stimulating hormone (FSH) level, and antral follicle count (AFC), and according to the cut-off values for cut-off points, the age, anti-Mullerian hormone (AMH) level, follicle-stimulating hormone (FSH) level, and antral follicle count (AFC) are converted into two-categorical variables, which are then used as predictive variables to calculate the probability (p) of poor ovarian response of the subject.

In the patent application CN202010265214.7, a system for evaluating the ovarian reserve function of a subject is provided, which comprises: a data collection module, which is used for acquiring data of the age, anti-Mullerian hormone (AMH) level, follicle-stimulating hormone (FSH) level; and a module for calculating the ovarian reserve function, which is used for calculating the information acquired in the data collection module, so as to calculate the probability (p) of poor ovarian response of the subject.

Although the above systems have been developed, blood collection in both systems needs to be done on a specific day of the menstrual cycle, since antral follicle count (AFC) requires a method of transvaginal sonography to count the total number of antral follicles in both ovaries, and follicle-stimulating hormone (FSH) of the subject requires to be measured using the venous blood of menstruate 2-4 days of the subject. Therefore, there is a need for further development of new systems, hoping to accurately predict the ovarian reserve function of subject by more simple and convenient inspection of data.

### SUMMARY

As mentioned above, judging the ovarian reserve function of a subject is a very important job for clinicians and the like. By evaluating the ovarian reserve function, the ovarian responsiveness of a patient can be predicted, which is an important clinical outcome during ovulation induction therapy. In the past, clinicians often made judgments based on age, body mass index, endocrine factor levels, and number of antral follicles in combination with their own experience, which were subject to a certain degree of subjectivity. The present system can accurately evaluate the quality of ovarian reserve function for subjects to be treated, so as to assist clinicians in formulating more targeted treatment schemes in subsequent treatment.

To sum up, it is known that the determinant of ovarian responsiveness is the ovarian reserve function, but the inventors of the present application thought reversely and used expected ovarian responsiveness to evaluate the ovarian reserve function. The inventors of the present application first obtained the expected probability of poor ovarian response according to the basic situation of the patient, and then grouped the ovarian reserve function of the subject according to the default ovarian reserve function grouping parameters pre-stored in the system. And thus the level of the ovarian reserve function of the subject was determined, and the level of ovarian reserve and ovarian youth index was evaluated.

In particular, the present application relates to the following:
1. A system for evaluating an ovarian reserve function of a subject, comprising:
a data collection module, which is used for acquiring data of age and an anti-Mullerian hormone (AMH) level of the subject; and
a module for calculating the ovarian reserve function, which is used for calculating the information acquired in the data collection module, so as to calculate a probability (p) of poor ovarian response of the subject.
2. The system of item 1, wherein in the module for calculating the ovarian reserve function, a ovarian youth index score is also calculated based on the probability (p) of poor ovarian response, wherein the ovarian youth index score is equal to 100*(1−p).
3. The system of item 2, further comprising:
a grouping module, wherein a default ovarian reserve function grouping parameter is pre-stored in the grouping module, and according to the grouping parameter, the calculated probability p of the poor ovarian response and the ovarian youth index score are grouped, so as to group a ovarian reserve level of the subject.
4. The system of any one of items 1 to 3, wherein in the module for calculating the ovarian reserve function, the probability (p) of poor ovarian response of the subject is calculated using a multiple categorical variable into which the data of the age and the anti-Mullerian hormone (AMH) level of the subject is converted.
5. The system of item 4, wherein the anti-Mullerian hormone (AMH) level refers to an anti-Mullerian hormone concentration in the venous blood of the female subject on any day.
6. The system of item 4 or 5, wherein in the module for calculating the ovarian reserve function, the age of the subject is converted into a six-categorical variable,
that is, the age of the subject is divided into six groups: the age of the subject is 30 years old and below, the age of the subject is greater than 30 years old and 35 years old and below, the age of the subject is greater than 35 years old and 37 years old and below, the age of the subject is greater than 37 years old and less than or equal to 39 years old, the age of the subject is greater than 39 years old and 42 years old and below, the age of the subject is greater than 42 years old.
7. The system of any one of items 4 to 6, wherein, in the module for calculating the ovarian reserve function, the anti-Mullerian hormone (AMH) level of the subject is converted into a ten-categorical variable,
that is, the anti-Mullerian hormone (AMH) level of the subject is divided into ten groups, namely: the anti-Mullerian hormone (AMH) level of the subject is less than 0.2 ng/ml, the anti-Mullerian hormone (AMH) level of the subject is greater than or equal to 0.2 ng/ml and less than 0.4 ng/ml, the anti-Mullerian hormone (AMH) level of the subject is greater than or equal to 0.4 ng/ml and less than 0.6 ng/ml, the anti-Mullerian hormone (AMH) level of the subject is greater than or equal to 0.6 ng/ml and less than 1.0 ng/ml, the anti-Mullerian hormone (AMH) level of the subject is greater than or equal to 1.0 ng/ml and less than 1.2 ng/ml, the anti-Mullerian hormone (AMH) level of the subject is greater than or equal to 1.2 ng/ml and less than 1.4 ng/ml, the anti-Mullerian hormone (AMH) level of the subject is greater than or equal to 1.4 ng/ml and less than 1.8 ng/ml, the anti-Mullerian hormone (AMH) level of the subject is greater than or equal to 1.8 ng/ml and less than 2 ng/ml, the anti-Mullerian hormone (AMH) level of the subject is greater than or equal to 2.0 ng/ml and less than 3.0 ng/ml, and the anti-Mullerian hormone (AMH) level of the subject is greater than or equal to 3.0 ng/ml.
8. The system of any one of items 1 to 7, wherein, a formula for predicting the probability (p) of poor ovarian response of the subject obtained by fitting based on the multiple categorical variable into which the data of the age and the anti-Mullerian hormone (AMH) level of the subject is converted is pre-stored in the module for calculating the ovarian reserve function.
9. The system of item 8, wherein the formula is the following formula I:

p=1/(1+e\hat{ }(−(a+b*age+c*AMH))) (Formula I)
wherein p is the calculated parameter for characterizing the ovarian reserve function of the subject,
wherein a, b, c are unitless parameters;
wherein in the module for calculating the ovarian reserve function, values of b and c are acquired based on the age and the anti-Mullerian hormone (AMH) level of the subject, and are brought into formula I for calculation, in the calculation, values of age and AMH are 0 or 1.
10. The system of item 9, wherein a is any value selected from 0.231877 to 1.0625987, preferably 0.6472378;
when the age of the subject is less than or equal to 30 years old, age is 0
when the age of the subject is greater than 30 years old and less than or equal to 35 years old, age is 1, and b is any value selected from 0.0078627 to 0.561014, preferably 0.2844384,
when the age of the subject is greater than 35 years old and less than or equal to 37 years old, age is 1, and b is any value selected from 0.095691 to 0.7641299, preferably 0.4299105,
when the age of the subject is greater than 37 years old and less than or equal to 39 years old, age is 1, and b is any value selected from 0.2368123 to 0.9401274, preferably 0.5884699,
when the age of the subject is greater than 39 years old and less than or equal to 42 years old, age is 1, and b is any value selected from 0.7081067 to 1.3787265, preferably 1.0434166,
when the age of the subject is greater than 42 years old, age is 1, and b is any value selected from 0.3552159 to 1.2587011, preferably 0.8069585,
when the anti-Mullerian hormone (AMH) level of the subject is less than 0.2 ng/ml, AMH is 0,
when the anti-Mullerian hormone (AMH) level of the subject is greater than or equal to 0.2 ng/ml and less than 0.4 ng/ml, AMH is 1, and c is any value selected from −1.044221 to −0.101256, preferably −0.572738,
when the anti-Mullerian hormone (AMH) level of the subject is greater than or equal to 0.4 ng/ml and less than 0.6 ng/ml, AMH is 1, and c is any value selected from −1.336186 to −0.412306, preferably −0.874246,
when the anti-Mullerian hormone (AMH) level of the subject is greater than or equal to 0.6 ng/ml and less than 1.0 ng/ml, AMH is 1, and c is any value selected from −2.095978 to −1.254203, preferably −1.675091,
when the anti-Mullerian hormone (AMH) level of the subject is greater than or equal to 1.0 ng/ml and less than 1.2 ng/ml, AMH is 1, and c is any value selected from −2.713953 to −1.750292, preferably is −2.232123,
when the anti-Mullerian hormone (AMH) level of the subject is greater than or equal to 1.2 ng/ml and less than 1.4 ng/ml, AMH is 1, and c is any value selected from −2.880223 to −1.882292, preferably −2.381258,
when the anti-Mullerian hormone (AMH) level of the subject is greater than or equal to 1.4 ng/ml and less than 1.8 ng/ml, AMH is 1, and c is any value selected from −3.363288 to −2.402785, preferably −2.883037,
when the anti-Mullerian hormone (AMH) level of the subject is greater than or equal to 1.8 ng/ml and less than 2 ng/ml, AMH is 1, and c is any value selected from −3.879955 to −2.589504, preferably −3.234729,
when the anti-Mullerian hormone (AMH) level of the subject is greater than or equal to 2.0 ng/ml and less than 3.0 ng/ml, AMH is 1, and c is any value selected from −4.296995 to −3.309204, preferably −3.803099, and
when the anti-Mullerian hormone (AMH) level of the subject is greater than or equal to 3.0 ng/ml, AMH is 1, and c is any value selected from −4.793561 to −3.876481, preferably −4.335021.
11. The system of any one of items 2 to 10, wherein a basis for grouping pre-stored in the grouping module is as follows:
when the calculated probability (p) of poor ovarian response of the subject is <10%, and the ovarian youth index score is >90 points, the grouping module determines that the subject has very good ovarian reserve function;
when the calculated probability (p) of poor ovarian response of the subject is ≥10% and <25%, and the ovarian youth index score is >75 to 90 points, the grouping module determines that the subject has good ovarian reserve function;
when the calculated probability (p) of poor ovarian response of the subject is ≥25% and <50%, and the ovarian youth index score is >50 to 75 points, the grouping module determines that the subject has poor ovarian reserve function;
when the calculated probability (p) of poor ovarian response of the subject is ≥50%, and the ovarian youth index score is ≤50 points, the grouping module determines that the subject has very poor ovarian reserve function.
12. A method for evaluating an ovarian reserve function of a subject, comprising:
a data collection step, for acquiring data of age and an anti-Mullerian hormone (AMH) level of the subject; and
a step for calculating the ovarian reserve function, for calculating the information acquired in the data collection step, so as to calculate a probability (p) of poor ovarian response of the subject.
13. The method of item 12, wherein in the step for calculating the ovarian reserve function, a ovarian youth index score is also calculated based on the probability (p) of poor ovarian response, wherein the ovarian youth index score is equal to 100*(1−p).
14. The method of item 13, further comprising:
a grouping step, in which the default ovarian reserve function grouping parameter is pre-stored, and according to the grouping parameter, the calculated probability p of the poor ovarian response and the ovarian youth index score are grouped, so as to group a ovarian reserve level of the subject.
15. The method of any one of items 12 to 14, wherein in the step for calculating the ovarian reserve function, the probability (p) of poor ovarian response of the subject is calculated using a multiple categorical variable into which the data of the age and the anti-Mullerian hormone (AMH) level of the subject is converted.
16. The method of item 15, wherein the anti-Mullerian hormone (AMH) level refers to an anti-Mullerian hormone concentration in venous blood of a female subject on any day.
17. The method of item 15 or 16, wherein in the step for calculating the ovarian reserve function, the age of the subject is converted into a six-categorical variable,
that is, the age of the subject is divided into six groups: the age of the subject is less than or equal to 30 years old, the age of the subject is greater than 30 years old and less than or equal to 35 years old, the age of the subject is greater than 35 years old and less than or equal to 37 years old, the age of the subject is greater than 37 years old and less than or equal to 39 years old, the age of the subject is greater than 39 years old and less than or equal to 42 years old, the age of the subject is greater than 42 years old.
18. The method of any one of items 15 to 17, wherein in the step for calculating the ovarian reserve function, the anti-Mullerian hormone (AMH) level of the subject is converted into a ten-categorical variable,
that is, the anti-Mullerian hormone (AMH) level of the subject is divided into ten groups, namely: the anti-Mullerian hormone (AMH) level of the subject is less than 0.2 ng/ml, the anti-Mullerian hormone (AMH) level of the subject is greater than or equal to 0.2 ng/ml and less than 0.4 ng/ml, the anti-Mullerian hormone (AMH) level of the subject is greater than or equal to 0.4 ng/ml and less than 0.6 ng/ml, the anti-Mullerian hormone (AMH) level of the subject is greater than or equal to 0.6 ng/ml and less than 1.0 ng/ml, the anti-Mullerian hormone (AMH) level of the subject is greater than or equal to 1.0 ng/ml and less than 1.2 ng/ml, the anti-Mullerian hormone (AMH) level of the subject is greater than or equal to 1.2 ng/ml and less than 1.4 ng/ml, the anti-Mullerian hormone (AMH) level of the subject is greater than or equal to 1.4 ng/ml and less than 1.8 ng/ml, the anti-Mullerian hormone (AMH) level of the subject is greater than or equal to 1.8 ng/ml and less than 2 ng/ml, the anti-Mullerian hormone (AMH) level of the subject is greater than or equal to 2.0 ng/ml and less than 3.0 ng/ml, and the anti-Mullerian hormone (AMH) level of the subject is greater than or equal to 3.0 ng/ml.
19. The method of any one of items 12 to 18, wherein in the step for calculating the ovarian reserve function, a formula for predicting the probability (p) of poor ovarian response of the subject obtained by fitting based on the multiple categorical variable into which the data of the age and the anti-Mullerian hormone (AMH) level of the subject is converted is pre-stored.
20. The method of item 19, wherein the formula is the following formula I:

$$p = 1 / ( 1 + e \hat{} ( - ( a + b * age + c * AMH ) ) ) \qquad ( Formula I )$$

wherein, p is the calculated parameter for characterizing the ovarian reserve function of the subject,
wherein, a, b, c are unitless parameters;
wherein, in the step for calculating the ovarian reserve function, values of b and c are acquired based on the age and the anti-Mullerian hormone (AMH) level of the subject, and are brought into formula I for calculation, in the calculation, values of age and AMH are 0 or 1.
21. The method of item 20, wherein a is any value selected from 0.231877 to 1.0625987, preferably 0.6472378;
when the age of the subject is less than or equal to 30 years old, age is 0
when the age of the subject is greater than 30 years old and less than or equal to 35 years old, age is 1, and b is any value selected from 0.0078627 to 0.561014, preferably 0.2844384,
when the age of the subject is greater than 35 years old and less than or equal to 37 years old, age is 1, and b is any value selected from 0.095691 to 0.7641299, preferably 0.4299105,
when the age of the subject is greater than 37 years old and less than or equal to 39 years old, age is 1, and b is any value selected from 0.2368123 to 0.9401274, preferably 0.5884699,
when the age of the subject is greater than 39 years old and less than or equal to 42 years old, age is 1, and b is any value selected from 0.7081067 to 1.3787265, preferably 1.0434166,
when the age of the subject is greater than 42 years old, age is 1, and b is any value selected from 0.3552159 to 1.2587011, preferably 0.8069585,
when the anti-Mullerian hormone (AMH) level of the subject is less than 0.2 ng/ml, AMH is 0,
when the anti-Mullerian hormone (AMH) level of the subject is greater than or equal to 0.2 ng/ml and less than 0.4 ng/ml, AMH is 1, and c is any value selected from −1.044221 to −0.101256, preferably −0.572738,
when the anti-Mullerian hormone (AMH) level of the subject is greater than or equal to 0.4 ng/ml and less than 0.6 ng/ml, AMH is 1, and c is any value selected from −1.336186 to −0.412306, preferably −0.874246,
when the anti-Mullerian hormone (AMH) level of the subject is greater than or equal to 0.6 ng/ml and less than 1.0 ng/ml, AMH is 1, and c is any value selected from −2.095978 to −1.254203, preferably −1.675091,
when the anti-Mullerian hormone (AMH) level of the subject is greater than or equal to 1.0 ng/ml and less than 1.2 ng/ml, AMH is 1, and c is any value selected from −2.713953 to −1.750292, preferably is −2.232123,
when the anti-Mullerian hormone (AMH) level of the subject is greater than or equal to 1.2 ng/ml and less than 1.4 ng/ml, AMH is 1, and c is any value selected from −2.880223 to −1.882292, preferably −2.381258,
when the anti-Mullerian hormone (AMH) level of the subject is greater than or equal to 1.4 ng/ml and less than 1.8 ng/ml, AMH is 1, and c is any value selected from −3.363288 to −2.402785, preferably −2.883037,
when the anti-Mullerian hormone (AMH) level of the subject is greater than or equal to 1.8 ng/ml and less than 2 ng/ml, AMH is 1, and c is any value selected from −3.879955 to −2.589504, preferably −3.234729,
when the anti-Mullerian hormone (AMH) level of the subject is greater than or equal to 2.0 ng/ml and less than 3.0 ng/ml, AMH is 1, and c is any value selected from −4.296995 to −3.309204, preferably −3.803099, and
when the anti-Mullerian hormone (AMH) level of the subject is greater than or equal to 3.0 ng/ml, AMH is 1, and c is any value selected from −4.793561 to −3.876481, preferably −4.335021.
22. The method of any one of items 13 to 21, wherein a basis for grouping pre-stored in the grouping step is as follows:
when the calculated probability (p) of poor ovarian response of the subject is <10%, and the ovarian youth index score is >90 points, the grouping module determines that the subject has very good ovarian reserve function;
when the calculated probability (p) of poor ovarian response of the subject is ≥10% and <25%, and the ovarian youth index score is >75 to 90 points, the grouping step determines that the subject has good ovarian reserve function;
when the calculated probability (p) of poor ovarian response of the subject is ≥25% and <50%, and the ovarian youth index score is >50 to 75 points, the grouping step determines that the subject has poor ovarian reserve function; and
when the calculated probability (p) of poor ovarian response of the subject is ≥50%, and the ovarian youth index score is ≤50 points, the grouping step determines that the subject has very poor ovarian reserve function.

### Effects of the Application

Decreased ovarian reserve is the main cause of female fertility decline. However, there are large individual differences in ovarian reserve. Some people face the risk of decrease or even depletion of ovarian reserve at a young age. Therefore, timely evaluation of ovarian reserve is very necessary. The evaluation of ovarian reserve function can help women of childbearing age understand their fertility status, so as to arrange their fertility plans reasonably. For women with a history of infertility, it can be used to predict ovarian responsiveness in women of childbearing age, and provide a reference for clinical diagnosis and treatment planning of infertility. At present, the main basis for the diagnosis of decreased ovarian reserve function at home and abroad is the diagnosis of poor ovarian response in Bologna criteria. Therefore, the indicators for evaluating ovarian reserve function are actually indicators for predicting ovarian responsiveness.

Specifically, in the present application, the system for evaluating the ovarian reserve function of a subject of the present application can be used to calculate the probability of poor ovarian response of the subject, and thus the ovarian reserve level of the subject is grouped based on the probability of poor ovarian response.

By using the system of the present application, the parameter (p) for predicting the probability of poor ovarian response of the subject can be calculated, and the ovarian reserve function of the subject can be grouped according to the default ovarian reserve function grouping parameters pre-stored in the system, so as to determine the level of ovarian reserve function thereof, so that the ovarian reserve level can be evaluated.

The inventors of the present application realized that ovarian responsiveness is closely related to ovarian reserve, and the poorer the ovarian reserve function is, the higher the risk of ovarian poor response is. Clinically, whether the ovarian poor response is high-risk is commonly used to evaluate the decline of ovarian reserve function. The order of ovarian reserve from high to low is the order of the probability of poor ovarian response from low to high.

By using the system and method of the present application, the ovarian reserve function of the subject to be treated can be accurately evaluated, and the clinician can be assisted in formulating a more targeted treatment scheme in the subsequent treatment. For ordinary women of childbearing age, especially women of childbearing age who want to give birth but are not sure when to give birth, the system and method of the present application can help them evaluate their own ovarian reserve and formulate a reasonable fertility plan.

Although there are existing methods and systems for evaluating ovarian reserve function with three or four indicators developed by the applicant, there is still a need to further develop simpler and more accurate models in the present art. The inventors of the invention used the serum AMH level and age of the subjects on any day to evaluate the ovarian reserve function for the first time. Compared with the previous system, fasting or not has no effect on the serum AMH level, so it is not necessary to collect blood at a specific time and under fasting, and the detection can be performed at any time, and its accuracy can still reach the level of the previous three-indicator and four-indicator systems. Since only the AMH level needs to be detected, and the detection of antral follicle count (AFC) and follicle-stimulating hormone (FSH) is omitted, the detection cost can be greatly reduced.

By using the system and method of the present application, the levels of the ovarian reserve and ovarian youth index of the subject can be evaluated fast, conveniently and accurately, solving the problems such as poor repeatability and inconsistent standards caused by evaluation of the ovarian reserve function mainly based on the doctor's experience and some simple cut-point values of the ovarian reserve indicators in the prior art. The ovarian youth index is displayed in the form of a percentage system, and the higher score indicates the better ovarian youth index, which also is easier to be understood and accepted by the public.

### DETAILED DESCRIPTION

Specific embodiments of the present application will be described in more detail below. It should be understood, however, that the present application may be implemented in various forms and should not be limited by the embodiments set forth herein. Rather, these embodiments are provided so that the present application will be more thoroughly understood, and the scope of the present application will fully be conveyed to those skilled in the art.

It should be noted that certain terms are used in the description and claims to refer to specific components. It should be understood by those skilled in the art that the same component may be referred to by different nouns. The present description and claims do not use the difference of nouns as a way of distinguishing components, but use the difference in function of the components as a criterion for distinguishing. As referred to throughout the description and claims, “comprising” or “including” is an open-ended term and should be interpreted as “including but not limited to”. Subsequent statements in the description are preferred embodiments for implementing the present application, however, the statements are for the purpose of general principles of the description and are not intended to limit the scope of the present application. The scope of protection of the present application should be determined by the appended claims.

In this application, ovarian reserve refers to the number of primordial follicles contained in the ovarian cortex, which is called ovarian reserve. It reflects the ability of the ovary to provide health, fertile eggs and is the most important assessment indicator of ovarian function in women. Generally speaking, the higher the number of primordial follicles, the better the quality and the higher the probability of conception.

However, the number of primordial follicles cannot be evaluated non-invasively. It can only be evaluated by the number of follicles mobilized in each menstrual cycle. Too few follicles mobilized in the IVF-ET cycle (poor ovarian response) suggest that the ovarian reserve function, that is, ovarian youth index is decreased.

The age factor is generally considered to be the most important factor in evaluating ovarian reserve. A study on age and IVF success rate showed that the IVF success rate was about 26% in women under the age of 30, while the IVF success rate was only 9% when the age was 37 years and older.

The mechanism by which ovarian reserve decreases with age is as follows. (1) The number of follicles decreases. Primordial follicles appear after the sex differentiation of the embryo. At this time, the number of follicles is the largest. From puberty, the follicles begin to develop and mature. With the completion of ovulation, a large number of follicles that are recruited but not expelled shrink and disappear to form the corpus luteum. The number of follicles continues to decrease with age: it is largest in the 20-week-old embryos in humans with about 6 million follicles, reduced to 700,000 to 2 million in the neonatal period, is about 40,000 at ovarian youth index, and is only a few thousand at the beginning of menopause, until it is completely exhausted. (2) The egg quality is decreased. Embryonic quality is mainly determined by egg quality. Older age can lead to increased risk of egg aneuploidy, increased risk of mitochondrial dysfunction, loss of egg polarity, and egg cell epigenetic changes. (3) Endocrine factor: the hypothalamus-pituitary-ovarian axis regulates women's menstrual cycle and ovulation, and abnormal endocrine levels of this axis can lead to infertility. AMH and inhibin B are secreted by small follicles and are a direct manifestation of the ability of ovarian reserve. The ovarian reserve decreases with age, the number of follicles that can be recruited decreases, and therefore the concentration of AMH and inhibin B secreted by it decreases. Inhibin B can negatively regulate pituitary FSH secretion, and a decrease in inhibin B level leads to an increase in FSH secretion in the luteal phase. The early increase in FSH promotes the growth of new follicles and E2 secretion, which ultimately shortens the menstrual cycle. Serum FSH level increases, inhibin B level decreases, and follicle sensitivity to FSH decreases, suggesting that the number of antral follicles which can be recruited is reduced. The menstrual cycle is the manifestation of ovarian reserve and fertility. The shortening of the menstrual cycle is caused by old age, and the reduction of the menstrual cycle by 2-3 days is a sensitive indicator of the aging of the reproductive system, indicating that the follicle growth is initiated in advance (the level of FSH is increased), and the primordial follicle reserve, that is ovarian youth index, decreased.

Continuous variables: in statistics, variables can be divided into continuous variables and categorical variables according to whether the value of the variable is continuous or not. A variable that can take any value within a certain interval is called a continuous variable. Its values are continuous, and between two adjacent values infinite division can be made, that is, infinite values can be taken. For example, the specifications and dimensions of production parts, anthropometric height, weight, chest circumference, etc. are continuous variables, and their values can only be obtained by measurement or metering methods. Conversely, those whose values can only be calculated in natural numbers or integer units are discrete variables. For example, the number of enterprises, the number of employees, the number of equipment, etc. can only be counted by the metering units. The value of this variable is generally obtained by counting methods.

Categorical variables are variables in terms of geographic location, demographics, etc. and are used to group survey respondents. Descriptive variables describe the difference between a certain customer group and other customer groups. Most categorical variables are also descriptive variables. Categorical variables can be divided into two categories: unordered categorical variables and ordered categorical variables. The unordered categorical variable means there is no difference in degree and order between the classified categories or attributes. It can be further divided into \textcircled{1} binary category, such as gender (male, female), drug reaction (negative and positive), etc.; \textcircled{2} multinary category, such as blood type (O, A, B, AB), occupation (workers, farmers, businessmen, students, soldiers), etc. The ordinal categorical variable has a difference in degree between the categories. For example, urine sugar test results are categorized by −, ±, +, ++, +++; curative effects are categorized by cure, obvious effect, improvement and no effect. For ordered categorical variables, firstly, they should be grouped in rank order, the number of observation units in each group should be counted, and the frequency table of ordinal variables (each rank) should be compiled. The obtained data are called rank data.

The types of variables are not invariable. According to the needs of research purposes, various types of variables can be converted each other. For example, the amount of hemoglobin (g/L) is originally a numerical variable. If it is divided into two categories according to normal and low hemoglobin, it can be analyzed by binomial data; if it is divided into five ranks according to severe anemia, moderate anemia, mild anemia, normal, and increased hemoglobin, it can be analyzed by ranked data. Sometimes categorical data can also be quantified, for example, the patient's nausea response can be expressed as 0, 1, 2, or 3, and it can be analyzed by numerical variable data (quantitative data).

The present application relates to a system for evaluating ovarian reserve function of a subject, comprising:
a data collection module, which is used for acquiring data of age and an anti-Mullerian hormone (AMH) level of the subject; and
a module for calculating the ovarian reserve function, which is used for calculating the information acquired in the data collection module, so as to calculate a probability (p) of poor ovarian response of the subject.

The present application also relates to a system for evaluating ovarian reserve function of a subject, comprising:
a data collection module, which is used for acquiring data of age and an anti-Mullerian hormone (AMH) level of the subject;
a module for calculating the ovarian reserve function, which is used for calculating the information acquired in the data collection module, so as to calculate a probability (p) of poor ovarian response of the subject; and
a grouping module, wherein a default ovarian reserve function grouping parameter is pre-stored in the grouping module, and according to the grouping parameter, the calculated probability p of poor ovarian response and the ovarian youth index score are grouped, so as to group a ovarian reserve level of the subject, wherein the ovarian youth index score is equal to 100*(1−p).

In the module for calculating the ovarian reserve function, the probability (p) of poor ovarian response of the subject is calculated using a multiple categorical variable into which the data of the age and the anti-Mullerian hormone (AMH) level of the subject is converted, and a ovarian youth index score is also calculated based on the probability (p) of poor ovarian response, wherein the ovarian youth index score is equal to 100*(1−p).

Anti-Mullerian hormone (AMH) is a hormone secreted by the granulosa cells of the ovarian small follicles. Female babies in the fetal period start to produce AMH from 36 weeks old. The grater the number of small follicles in the ovary, the higher the concentration of AMH. On the contrary, when the follicles are gradually consumed with age and various factors, the AMH concentration will also decrease accordingly, and the closer to the menopause, the AMH will gradually tend to 0.

The anti-Mullerian hormone (AMH) level in the present application refers to the anti-Mullerian hormone concentration in a serum sample of the venous blood of the subject on any day.

In the module for calculating the ovarian reserve function, the age of the subject is converted into a six-categorical variable, that is, the age of the subject is divided into six groups, namely: the age of the subject is less than or equal to 30 years old, the age of the subject is greater than 30 years old and less than or equal to 35 years old, the age of the subject is greater than 35 years old and less than or equal to 37 years old, the age of the subject is greater than 37 years old and less than or equal to 39 years old, the age of the subject is greater than 39 years old and less than or equal to 42 years old, and the age of the subject is greater than 42 years old.

In the module for calculating the ovarian reserve function, the inventors of the present application have conducted intensive research to convert the anti-Mullerian hormone (AMH) level of the subject into a ten-categorical variable, that is, the anti-Mullerian hormone (AMH) level is divided into ten groups, namely: the anti-Mullerian hormone (AMH) level of the subject is less than 0.2 ng/ml, the anti-Mullerian hormone (AMH) level of the subject is greater than or equal to 0.2 ng/ml and less than 0.4 ng/ml, the anti-Mullerian hormone (AMH) level of the subject is greater than or equal to 0.4 ng/ml and less than 0.6 ng/ml, the anti-Mullerian hormone (AMH) level of the subject is greater than or equal to 0.6 ng/ml and less than 1.0 ng/ml, the anti-Mullerian hormone (AMH) level of the subject is greater than or equal to 1.0 ng/ml and less than 1.2 ng/ml, the anti-Mullerian hormone (AMH) level of the subject is greater than or equal to 1.2 ng/ml and less than 1.4 ng/ml, the anti-Mullerian hormone (AMH) level of the subject is greater than or equal to 1.4 ng/ml and less than 1.8 ng/ml, the anti-Mullerian hormone (AMH) level of the subject is greater than or equal to 1.8 ng/ml and less than 2 ng/ml, the anti-Mullerian hormone (AMH) level of the subject is greater than or equal to 2.0 ng/ml and less than 3.0 ng/ml, and the anti-Mullerian hormone (AMH) level of the subject is greater than or equal to 3.0 ng/ml.

In the module for calculating the ovarian reserve function, the applicant of the present application have carefully studied, and as described above, divide the age of the subject into the six-categorical variable and divide the anti-Mullerian hormone (AMH) level into the ten-categorical variable. They are brought into a categorical variable model to calculate the probability of poor ovarian response. The ovarian reserve function is grouped according to the grouping principle summarized by the inventors of this application to obtain the situation of the ovarian reserve function of the subject.

By converting the above two variables into different multiple categorical variables and using such multiple categorical variables for data analysis, the ovarian reserve function of subject can be more accurately predicted, and the model is more stable. In the present application, the two indicators of age and anti-Mullerian hormone (AMH) level are used to construct a system for predicting ovarian reserve, which can replace the original system for predicting ovarian reserve constructed by using the three indicators of age, anti-Mullerian hormone (AMH) level, follicle-stimulating hormone (FSH) level. Although the prediction effect of the original three-indicator system is very good, the convenience of the system can be further improved and the cost of operating the entire system can be reduced if the need to acquire data of follicle-stimulating hormone (FSH) level at specific times can be avoided. In addition, this application also classifies the two indicators of age and anti-Mullerian hormone (AMH) level in more detail and optimizes the classification criteria to convert age into a six-categorical variable and convert anti-Mullerian hormone (AMH) level into a ten-categorical variable, thereby realizing the replacement of the original three-indicator system with two-indicator system and achieving the same good prediction effect.

Using the system of the present application can help clinicians formulate a more effective ovulation induction scheme, and improve pregnancy rate or reduce treatment cost by accurately evaluating the ovarian reserve function and ovarian youth index of the subject.

In the module for calculating the ovarian reserve function, a formula for predicting the probability (p) of poor ovarian response of the subject obtained by fitting based on the multi-categorical variable into which the data of the age and the anti-Mullerian hormone (AMH) level of the subject in the existing database is converted is pre-stored. And according to the grouping criteria, the ovarian reserve function of the subject is grouped.

In the present application, the existing database refers to the available database composed of subjects who are receiving treatment or have previously received treatment and meet the following inclusion and exclusion criteria. There is no agreement on the sample size of the database. Of course, the more the sample size of the database, the better, for example, the sample size of the database may be 100 subjects, 200 subjects, 300 subjects, preferably 400 subjects or more, more preferably 500 subjects or more. In a specific embodiment, an existing database consisting of 4796 samples is used.

The above inclusion and exclusion criteria are as follows, respectively. Inclusion criteria: women aged between 20 and 45 years, with a body mass index (BMI) ≤30, six consecutive menstrual cycles of 25 to 45 days, normal bilateral ovarian morphology evaluated by transvaginal ultrasound, and number of previous IVF/ICSI-ET cycles ≤2. Exclusion criteria: hydrosalpinx, unilateral ovarian AFC >20, polycystic ovary syndrome, other untreated metabolic or endocrine disorders, previous surgery for the ovaries or uterine cavity, intrauterine abnormalities, pregnancy within 3 months, smoking, use of oral contraceptive or other hormones in the previous 2 months, previous experience with radiation therapy or chemotherapy, couples undergoing genetic diagnosis with PGD (preimplantation Genetic Diagnosis)/PGS (preimplantation genetic screening) treatment.

When selecting samples for the database, subjects who can be included in the database need to meet both the above inclusion and exclusion criteria.

The module for calculating the ovarian reserve function uses the following formula to calculate a parameter (p) characterizing the ovarian reserve function of the subject from the data acquired in the data collection module:

$$p = 1 / ( 1 + e \hat{} ( - ( a + b * age + c * AMH ) ) ) \qquad ( Formula I )$$

wherein p is the calculated parameter for characterizing the ovarian reserve function of the subject,
wherein a, b, and c are unitless parameters.
wherein in the module for calculating the ovarian reserve function, values of b, c and d are acquired based on the age, the anti-Mullerian hormone (AMH) level, and the follicle-stimulating hormone (FSH) level of the subject, and are brought into formula I for calculation, in the calculation, values of age and AMH are 0 or 1.

Further, a is any value selected from 0.231877 to 1.0625987, preferably 0.6472378; when the age of the subject is less than or equal to 30 years old, age is 0; when the age of the subject is greater than 30 years old and less than or equal to 35 years old, age is 1, and b is any value selected from 0.0078627 to 0.561014, preferably 0.2844384, when the age of the subject is greater than 35 years old and less than or equal to 37 years old, age is 1, and b is any value selected from 0.095691 to 0.7641299, preferably 0.4299105; when the age of the subject is greater than 37 years old and less than or equal to 39 years old, age is 1, and b is any value selected from 0.2368123 to 0.9401274, preferably 0.5884699; when the age of the subject is greater than 39 years old and less than or equal to 42 years old, age is 1, and b is any value selected from 0.7081067 to 1.3787265, preferably 1.0434166; when the age of the subject is greater than 42 years old, age is 1, and b is any value selected from 0.3552159 to 1.2587011, preferably 0.8069585; when the anti-Mullerian hormone (AMH) level of the subject is less than 0.2 ng/ml, AMH is 0; when the anti-Mullerian hormone (AMH) level of the subject is greater than or equal to 0.2 ng/ml and less than 0.4 ng/ml, AMH is 1, and c is any value selected from −1.044221 to −0.101256, preferably −0.572738; when the anti-Mullerian hormone (AMH) level of the subject is greater than or equal to 0.4 ng/ml and less than 0.6 ng/ml, AMH is 1, and c is any value selected from −1.336186 to −0.412306, preferably −0.874246; when the anti-Mullerian hormone (AMH) level of the subject is greater than or equal to 0.6 ng/ml and less than 1.0 ng/ml, AMH is 1, and c is any value selected from −2.095978 to −1.254203, preferably −1.675091; when the anti-Mullerian hormone (AMH) level of the subject is greater than or equal to 1.0 ng/ml and less than 1.2 ng/ml, AMH is 1, and c is any value selected from −2.713953 to −1.750292, preferably is −2.232123; when the anti-Mullerian hormone (AMH) level of the subject is greater than or equal to 1.2 ng/ml and less than 1.4 ng/ml, AMH is 1, and c is any value selected from −2.880223 to −1.882292, preferably −2.381258; when the anti-Mullerian hormone (AMH) level of the subject is greater than or equal to 1.4 ng/ml and less than 1.8 ng/ml, AMH is 1, and c is any value selected from −3.363288 to −2.402785, preferably −2.883037; when the anti-Mullerian hormone (AMH) level of the subject is greater than or equal to 1.8 ng/ml and less than 2 ng/ml, AMH is 1, and c is any value selected from −3.879955 to −2.589504, preferably −3.234729; when the anti-Mullerian hormone (AMH) level of the subject is greater than or equal to 2.0 ng/ml and less than 3.0 ng/ml, AMH is 1, and c is any value selected from −4.296995 to −3.309204, preferably −3.803099; and when the anti-Mullerian hormone (AMH) level of the subject is greater than or equal to 3.0 ng/ml, AMH is 1, and c is any value selected from −4.793561 to −3.876481, preferably −4.335021.

Further, calculating the female ovarian youthfulness index according to the probability (p) of poor ovarian response of the subject is further comprised in the system in the present application. The ovary is the foundation of women, and the female ovarian youth index can represent the degree of female ovarian health. The higher the female ovarian youth index, the healthier the ovary. The value of ovarian youth level is equal to 100*(1−p). For example, when the probability p of the poor ovarian response is 0.1, the ovarian youth level is calculated as 100*(1−p), and the result is 90.

A basis for evaluating and grouping the ovarian reserve function is pre-stored in the grouping module of this application. When the calculated probability (p) of poor ovarian response of the subject is <10%, and the ovarian youth index score is >90 points, the grouping module determines that the subject has very good ovarian reserve function; when the calculated probability (p) of poor ovarian response of the subject is ≥10% and <25%, and the ovarian youth index score is 75 to 90 points, the grouping module determines that the subject has good ovarian reserve function; when the calculated probability (p) of poor ovarian response of the subject is ≥25% and <50%, and the ovarian youth index score is 50 to 75 points, the grouping module determines that the subject has poor ovarian reserve function; when the calculated probability (p) of poor ovarian response of the subject is ≥50%, and the ovarian youth index score is ≤50 points, the grouping module determines that the subject has very poor ovarian reserve function.

In another specific embodiment of the present application, the present application also relates to a method for evaluating ovarian reserve function of a subject, the method comprising a data collection step, for acquiring age and an anti-Mullerian hormone (AMH) level of the subject; and a step for calculating the ovarian reserve function, for calculating the information acquired in the data collection step, so as to calculate a probability (p) of poor ovarian response of the subject. In addition, the method further comprising: a grouping step, in which a pre-known ovarian reserve function grouping parameter is used, and according to the grouping parameter, the calculated probability p of poor ovarian response and the ovarian youth index score are grouped, so as to group the ovarian reserve level of the subject.

As mentioned above, the specific contents of the steps performed in the method of the present application, including the acquisition, grouping and processing mode of the data of the age and the anti-Mullerian hormone (AMH) level can refer to the steps performed in each module of the above-mentioned system involved in this application.

### EXAMPLE

Inclusion criteria: women aged between women aged between 20 and 45 years, with a body mass index (BMI)≤30, six consecutive menstrual cycles of 25 to 45 days, normal bilateral ovarian morphology evaluated by transvaginal ultrasound, and number of previous IVF/ICSI-ET cycles ≤2.

Exclusion criteria: hydrosalpinx, unilateral ovarian AFC>20, polycystic ovary syndrome, other untreated metabolic or endocrine disorders, previous surgery for the ovaries or uterine cavity, intrauterine abnormalities, pregnancy within 3 months, smoking, use of oral contraceptive or other hormones in the previous 2 months, previous experience with radiation therapy or chemotherapy, couples undergoing genetic diagnosis with PGD (preimplantation genetic diagnosis)/PGS (preimplantation genetic screening) treatment.

### Controlled Ovarian Stimulation (COS) Therapy

Gn (i.e, human recombinant FSH) treatment was initiated on day 2 or 3 in the menstrual cycle. The starting dose was selected based on the age, BMI (that is, body mass index, which is a number obtained by dividing the weight in kilograms by the square of the height in meters, which is a standard commonly used internationally to measure the fat degree of human body and whether it is healthy), and FSH and AFC levels of menstruation 2-4 days. During ovulation induction, the starting dose of Gn was adjusted based on ultrasound observation and serum E2 level. GnRH antagonist treatment began on day 5-7 of stimulation, when growing follicles were 10-12 mm in diameter. When at least 2 dominant follicles (≥18 mm in diameter) were visible by ultrasound, 5000-10000 IU of hCG was administered to initiate final oocyte maturation. Egg retrieval was performed 36 hours after administration of hCG. 1-3 embryos were transplanted or the embryos were cryopreserved. The progesterone support was then provided in luteal phase.

In the examples of this application, the applicant of this application used the subjects who received the above-mentioned GnRH antagonist treatment in the years 2017-2018, wherein finally 4796 subjects whose data met the above-mentioned criteria were included in this example, and used to construct the system involved in this application.

### Sample Acquisition and Endocrine Assay

For 4796 subjects as described above, venous blood samples were drawn and immediately inverted five times to promote complete blood clotting, and serum was collected by centrifugation and used for endocrine assessment. The follicle-stimulating hormone (FSH) level of the subject was measured on day 2 in the subject's menstrual cycle, and the anti-Mullerian hormone (AMH) level of the subject was measured on any day in the subject's menstrual cycle. Measurement of FSH in serum was performed using an Immulite 2000 immunoassay system (Siemens Healthineers, Shanghai, China). Quality control for the FSH assay was provided by Bio-RAD Laboratories (Lyphochek Immunoassay Plus Control, Trilevel, Cat. No. 370, Lot No. 40340). Serum AMH concentrations of the subject were detected using an ultrasensitive two-point ELISA kit (Ansh Labs, USA).

The AMH level on any day refers to the anti-Mullerian hormone level measured in the serum sample of venous blood of a female subject at any time, and fasting or no fasting has no effect on the AMH level. The data of the system used to build the model is shown in Table 1 below.

TABLE 1
Clinical and biochemical data of subjects
treated with GnRH antagonists

| | Data of 2017-2018 | Data of 2019 |
|---|---|---|
| | (n = 4796) | (n = 5009) |
| Average age (years old) | 32.9 ± 5.0 | 32.6 ± 4.7 |
| AMH (ng/ml) | 2.5 (1.2-4.5) | 2.5 (1.3-4.6) |

### Construction of System Model

In this example, the poor ovarian response and oocytes of less than 5 (specifically 0, 1, 2, 3 or 4) of the above 4796 subjects were defined as outcome variables, and the predictive variables were age and AMH level. In this example, the prediction model was constructed using the data of 2017-2018, that is, the data of 4,796 subjects was used to initially construct the model system of this application, and the data of 2019, namely, data of 5,009 subjects was used to verify the effectiveness of the system model. In the data of 2019, the subjects were all patients who received the above standard GnRH antagonist treatment, in which subjects with endocrine-related abnormal diseases and ovarian-related abnormal diseases were not excluded.

The specific steps were: using JMP Pro 14.2 software, firstly, multiple forward selection with 5-fold cross-validation was applied to the modeling data to construct a prediction model of poor ovarian response, and the effect of the model was verified by the validation data. The performance of the established prediction models was assessed using the area under the curve (AUC), sensitivity, specificity and provided in the software.

Multivariate logistic regression was first performed on the modeling data, i.e., the data of 4796 subjects, with the poor ovarian response or not as the outcome variable and the age and AMH as independent variables. Due to the strong correlation between the two independent variables, the two continuous variables were converted into categorical variables, and the criteria for grouping the two parameters, age and AMH level, were shown in Table 2.

TABLE 2
Basis for grouping

| | Grouping of the AMH (ng/ml) | Grouping of the age (years old) |
|---|---|---|
| 0 | <=0.2 | <=30 |
| 1 | <=0.4 | <=35 |
| 2 | <=0.6 | <=37 |
| 3 | <=1.0 | <=39 |
| 4 | <=1.2 | <=42 |
| 5 | <=1.4 | >42 |
| 6 | <=1.8 | |
| 7 | <=2 | |
| 8 | <=3 | |
| 9 | >3 | |

According to the grouping in Table 2, the ages of the subjects was divided into six groups, namely: the age of the subject is less than or equal to 30 years old, the age of the subject is greater than 30 years old and less than or equal to 35 years old, the age of the subject is greater than 35 years old and less than or equal to 37 years old, the age of the subject is greater than 37 years old and less than or equal to 39 years old, the age of the subject is greater than 39 years old and less than or equal to 42 years old, the age of the subject is greater than 42 years old. The anti-Mullerian hormone (AMH) level of the subject was divided into ten groups, namely: the anti-Mullerian hormone (AMH) level of the subject is less than 0.2 ng/ml, the anti-Mullerian hormone (AMH) level of the subject is greater than or equal to 0.2 ng/ml and less than 0.4 ng/ml, the anti-Mullerian hormone (AMH) level of the subject is greater than or equal to 0.4 ng/ml and less than 0.6 ng/ml, the anti-Mullerian hormone (AMH) level of the subject is greater than or equal to 0.6 ng/ml and less than 1.0 ng/ml, the anti-Mullerian hormone (AMH) level of the subject is greater than or equal to 1.0 ng/ml and less than 1.2 ng/ml, the anti-Mullerian hormone (AMH) level of the subject is greater than or equal to 1.2 ng/ml and less than 1.4 ng/ml, the anti-Mullerian hormone (AMH) level of the subject is greater than or equal to 1.4 ng/ml and less than 1.8 ng/ml, the anti-Mullerian hormone (AMH) level of the subject is greater than or equal to 1.8 ng/ml and less than 2 ng/ml, the anti-Mullerian hormone (AMH) level of the subject is greater than or equal to 2.0 ng/ml and less than 3.0 ng/ml, and the anti-Mullerian hormone (AMH) level of the subject is greater than or equal to 3.0 ng/ml.

At the same time, using the data of the above training group, the following formula was obtained by fitting and the parameters involved in the formula were confirmed, as shown in Table 3:

$$p = 1 / ( 1 + e \hat{} ( - ( a + b * age + c * AMH ) ) ) \qquad ( Formula I )$$

TABLE 3

| | Estimated | Standard | | |
|---|---|---|---|---|
| Variable | value | deviation | Wald χ² | P value |
| Intercept | 0.6472378 | 0.2119227 | 9.3276619 | 0.0023* |
| AMH [9 vs 0] | −4.335 | 0.2340 | 343.3393 | <.0001 |
| AMH [8 vs 0] | −3.8031 | 0.2520 | 227.7726 | <.0001 |
| AMH [7 vs 0] | −3.2347 | 0.3292 | 96.5493 | <.0001 |
| AMH [6 vs 0] | −2.883 | 0.2450 | 138.4393 | <.0001 |
| AMH [5 vs 0] | −2.3813 | 0.2546 | 87.4919 | <.0001 |
| AMH [4 vs 0] | −2.2321 | 0.2458 | 82.4412 | <.0001 |
| AMH [3 vs 0] | −1.6751 | 0.2147 | 60.8473 | <.0001 |
| AMH [2 vs 0] | −0.8742 | 0.2357 | 13.7592 | 0.0002 |
| AMH [1 vs 0] | −0.5727 | 0.2406 | 5.6686 | 0.0173 |
| Age [5 vs 0] | 0.807 | 0.2305 | 12.2579 | 0.0005 |
| Age [4 vs 0] | 1.0434 | 0.1711 | 37.1979 | <.0001 |
| Age [3 vs 0] | 0.5885 | 0.1794 | 10.7573 | 0.0010 |
| Age [2 vs 0] | 0.4299 | 0.1705 | 6.3561 | 0.0117 |
| Age [1 vs 0] | 0.2844 | 0.1411 | 4.0630 | 0.0438 |

As shown in formula I, p is a calculated parameter for characterizing the ovarian reserve function of the subject, wherein a, b, and c are unitless parameters; wherein in the module for calculating the ovarian reserve function, values of b and c are acquired based on the age and the anti-Mullerian hormone (AMH) level of the subject, and are brought into formula I for calculation, in the calculation, values of age and AMH are 0 or 1.

As shown in Table 3, the parameters involved in formula I are as follows. a is any value selected from 0.231877 to 1.0625987, preferably 0.6472378; when the age of the subject is less than or equal to 30 years old, age is 0; when the age of the subject is greater than 30 years old and less than or equal to 35 years old, age is 1, and b is any value selected from 0.0078627 to 0.561014, preferably 0.2844384; when the age of the subject is greater than 35 years old and less than or equal to 37 years old, age is 1, and b is any value selected from 0.095691 to 0.7641299, preferably 0.4299105; when the age of the subject is greater than 37 years old and less than or equal to 39 years old, age is 1, and b is any value selected from 0.2368123 to 0.9401274, preferably 0.5884699; when the age of the subject is greater than 39 years old and less than or equal to 42 years old, age is 1, and b is any value selected from 0.7081067 to 1.3787265, preferably 1.0434166; when the age of the subject is greater than 42 years old, age is 1, and b is any value selected from 0.3552159 to 1.2587011, preferably 0.8069585; when the anti-Mullerian hormone (AMH) level of the subject is less than 0.2 ng/ml, AMH is 0; when the anti-Mullerian hormone (AMH) level of the subject is greater than or equal to 0.2 ng/ml and less than 0.4 ng/ml, AMH is 1, and c is any value selected from −1.044221 to −0.101256, preferably −0.572738; when the anti-Mullerian hormone (AMH) level of the subject is greater than or equal to 0.4 ng/ml and less than 0.6 ng/ml, AMH is 1, and c is any value selected from −1.336186 to −0.412306, preferably −0.874246; when the anti-Mullerian hormone (AMH) level of the subject is greater than or equal to 0.6 ng/ml and less than 1.0 ng/ml, AMH is 1, and c is any value selected from −2.095978 to −1.254203, preferably −1.675091; when the anti-Mullerian hormone (AMH) level of the subject is greater than or equal to 1.0 ng/ml and less than 1.2 ng/ml, AMH is 1, and c is any value selected from −2.713953 to −1.750292, preferably is −2.232123; when the anti-Mullerian hormone (AMH) level of the subject is greater than or equal to 1.2 ng/ml and less than 1.4 ng/ml, AMH is 1, and c is any value selected from −2.880223 to −1.882292, preferably −2.381258; when the anti-Mullerian hormone (AMH) level of the subject is greater than or equal to 1.4 ng/ml and less than 1.8 ng/ml, AMH is 1, and c is any value selected from −3.363288 to −2.402785, preferably −2.883037; when the anti-Mullerian hormone (AMH) level of the subject is greater than or equal to 1.8 ng/ml and less than 2 ng/ml, AMH is 1, and c is any value selected from −3.879955 to −2.589504, preferably −3.234729; when the anti-Mullerian hormone (AMH) level of the subject is greater than or equal to 2.0 ng/ml and less than 3.0 ng/ml, AMH is 1, and c is any value selected from −4.296995 to −3.309204, preferably −3.803099; and when the anti-Mullerian hormone (AMH) level of the subject is greater than or equal to 3.0 ng/ml, AMH is 1, and c is any value selected from −4.793561 to −3.876481, preferably −4.335021. In order to verify the accuracy of the system, we compared the two-indicator, three-indicator, and four-indicator models in the same population (data of 2017-2018), as shown in Table 4. It can be seen from the results that the system constructed in this example and the system in the prior application can achieve the same evaluation level.

TABLE 4
Comparison of the effects of the three
models in the data of 2017-2018

| | | Four-indicator | Three-indicator |
|---|---|---|---|
| | System in this | system in the prior | system in the prior |
| | example | application | application |
| | 4796 subjects of | (CN201811516206.4) | (CN202010265214.7) |
| | training group | 3273 subjects of | 3273 subjects of |
| | (data of | verification group | verification group |
| | 2017-2018) | (data of 2017-2018) | (data of 2017-2018 |
| | (95% CI) | (95% CI) | (95% CI) |
| AUC | 0.854 (0.835 to | 0.838 (0.818 to 0.855) | 0.850 (0.832 to 0.867) |
| | 0.871) | | |

Therefore, according to the above formula I, the probability of poor ovarian response of a subject can be calculated based on the age and the concentration of anti-Mullerian hormone on any day in the menstrual cycle of the subject.

The population was grouped according to the calculated probability of poor ovarian response, and the grouping method adopted the grouping standard confirmed previously by the applicant (see CN201811516206.4), that is,
when the calculated probability (p) of poor ovarian response of the subject is <10%, and the ovarian youth index score is >90 points, the grouping module determines that the subject has very good ovarian reserve function; when the calculated probability (p) of poor ovarian response of the subject is ≥10% and <25%, and the ovarian youth index score is 75 to 90 points, the grouping module determines that the subject has good ovarian reserve function; when the calculated probability (p) of poor ovarian response of the subject is ≥25% and <50%, and the ovarian youth index score is 50 to 75 points, the grouping module determines that the subject has poor ovarian reserve function; when the calculated probability (p) of poor ovarian response of the subject is ≥50%, and the ovarian youth index score is ≤50 points, the grouping module determines that the subject has very poor ovarian reserve function.

Although the embodiments of the present application are described above, the present application is not limited to the above-mentioned specific embodiments and application fields. The above-mentioned specific embodiments are only illustrative, instructive, and not restrictive. Those of ordinary skill in the art can also make many forms under the inspiration of this specification and without departing from the scope of protection of the claims of the present application, which all belong to the protection of the present application.

1-11. (canceled)

12. A method for evaluating ovarian reserve function of a subject, comprising:
a data collection step, for acquiring data of age and an anti-Mullerian hormone (AMH) level of the subject; and
a step for calculating the ovarian reserve function, for calculating the information acquired in the data collection step, so as to calculate probability (p) of poor ovarian response of the subject.

13. The method of claim 12, wherein in the step for calculating the ovarian reserve function, a ovarian youth index score is also calculated based on the probability (p) of poor ovarian response, wherein the ovarian youth index score is equal to 100*(1−p).

14. The method of claim 13, further comprising:
a grouping step, in which the default ovarian reserve function grouping parameter is pre-stored, and according to the grouping parameter, the calculated probability p of the poor ovarian response and the ovarian youth index score are grouped, so as to group a ovarian reserve level of the subject.

15. The method of claim 12, wherein in the step for calculating the ovarian reserve function, the probability (p) of poor ovarian response of the subject is calculated using a multiple categorical variable into which the data of the age and the anti-Mullerian hormone (AMH) level of the subject is converted.

16. The method of claim 15, wherein the anti-Mullerian hormone (AMH) level refers to an anti-Mullerian hormone concentration in venous blood of a female subject on any day.

17. The method of claim 15, wherein in the step for calculating the ovarian reserve function, the age of the subject is converted into a six-categorical variable,
that is, the age of the subject is divided into six groups: the age of the subject is less than or equal to 30 years old, the age of the subject is greater than 30 years old and less than or equal to 35 years old, the age of the subject is greater than 35 years old and less than or equal to 37 years old, the age of the subject is greater than 37 years old and less than or equal to 39 years old, the age of the subject is greater than 39 years old and less than or equal to 42 years old, and the age of the subject is greater than 42 years old.

18. The method of claim 15, wherein in the step for calculating the ovarian reserve function, the anti-Mullerian hormone (AMH) level of the subject is converted into a ten-categorical variable,
that is, the anti-Mullerian hormone (AMH) level of the subject is divided into ten groups: the anti-Mullerian hormone (AMH) level of the subject is less than 0.2 ng/ml, the anti-Mullerian hormone (AMH) level of the subject is greater than or equal to 0.2 ng/ml and less than 0.4 ng/ml, the anti-Mullerian hormone (AMH) level of the subject is greater than or equal to 0.4 ng/ml and less than 0.6 ng/ml, the anti-Mullerian hormone (AMH) level of the subject is greater than or equal to 0.6 ng/ml and less than 1.0 ng/ml, the anti-Mullerian hormone (AMH) level of the subject is greater than or equal to 1.0 ng/ml and less than 1.2 ng/ml, the anti-Mullerian hormone (AMH) level of the subject is greater than or equal to 1.2 ng/ml and less than 1.4 ng/ml, the anti-Mullerian hormone (AMH) level of the subject is greater than or equal to 1.4 ng/ml and less than 1.8 ng/ml, the anti-Mullerian hormone (AMH) level of the subject is greater than or equal to 1.8 ng/ml and less than 2 ng/ml, the anti-Mullerian hormone (AMH) level of the subject is greater than or equal to 2.0 ng/ml and less than 3.0 ng/ml, and the anti-Mullerian hormone (AMH) level of the subject is greater than or equal to 3.0 ng/ml.

19. The method of claim 12, wherein in the step for calculating the ovarian reserve function, a formula for predicting the probability (p) of poor ovarian response of the subject obtained by fitting based on the multiple categorical variable into which the data of the age and the anti-Mullerian hormone (AMH) level of the subject is converted is pre-stored.

20. The method of claim 19, wherein the formula is the following formula I:
$$p = 1 / ( 1 + e \hat{} ( - ( a + b * age + c * AMH ) ) ) \qquad ( Formula I )$$
wherein p is the calculated parameter for characterizing the ovarian reserve function of the subject,
wherein a, b, c are unitless parameters;
wherein in the step for calculating the ovarian reserve function, values of b and c are acquired based on the age and the anti-Mullerian hormone (AMH) level of the subject, and are brought into formula I for calculation, in the calculation, values of age and AMH are 0 or 1.

21. The method of claim 20, wherein a is any value selected from 0.231877 to 1.0625987, preferably 0.6472378;
when the age of the subject is less than or equal to 30 years old, age is 0,
when the age of the subject is greater than 30 years old and less than or equal to 35 years old, age is 1, and b is any value selected from 0.0078627 to 0.561014, preferably 0.2844384,
when the age of the subject is greater than 35 years old and less than or equal to 37 years old, age is 1, and b is any value selected from 0.095691 to 0.7641299, preferably 0.4299105,
when the age of the subject is greater than 37 years old and less than or equal to 39 years old, age is 1, and b is any value selected from 0.2368123 to 0.9401274, preferably 0.5884699,
when the age of the subject is greater than 39 years old and less than or equal to 42 years old, age is 1, and b is any value selected from 0.7081067 to 1.3787265, preferably 1.0434166,
when the age of the subject is greater than 42 years old, age is 1, and b is any value selected from 0.3552159 to 1.2587011, preferably 0.8069585,
when the anti-Mullerian hormone (AMH) level of the subject is less than 0.2 ng/ml, AMH is 0,
when the anti-Mullerian hormone (AMH) level of the subject is greater than or equal to 0.2 ng/ml and less than 0.4 ng/ml, AMH is 1, and c is any value selected from −1.044221 to −0.101256, preferably −0.572738,
when the anti-Mullerian hormone (AMH) level of the subject is greater than or equal to 0.4 ng/ml and less than 0.6 ng/ml, AMH is 1, and c is any value selected from −1.336186 to −0.412306, preferably −0.874246,
when the anti-Mullerian hormone (AMH) level of the subject is greater than or equal to 0.6 ng/ml and less than 1.0 ng/ml, AMH is 1, and c is any value selected from −2.095978 to −1.254203, preferably −1.675091,
when the anti-Mullerian hormone (AMH) level of the subject is greater than or equal to 1.0 ng/ml and less than 1.2 ng/ml, AMH is 1, and c is any value selected from −2.713953 to −1.750292, preferably is −2.232123,
when the anti-Mullerian hormone (AMH) level of the subject is greater than or equal to 1.2 ng/ml and less than 1.4 ng/ml, AMH is 1, and c is any value selected from −2.880223 to −1.882292, preferably −2.381258,
when the anti-Mullerian hormone (AMH) level of the subject is greater than or equal to 1.4 ng/ml and less than 1.8 ng/ml, AMH is 1, and c is any value selected from −3.363288 to −2.402785, preferably −2.883037,
when the anti-Mullerian hormone (AMH) level of the subject is greater than or equal to 1.8 ng/ml and less than 2 ng/ml, AMH is 1, and c is any value selected from −3.879955 to −2.589504, preferably −3.234729,
when the anti-Mullerian hormone (AMH) level of the subject is greater than or equal to 2.0 ng/ml and less than 3.0 ng/ml, AMH is 1, and c is any value selected from −4.296995 to −3.309204, preferably −3.803099, and
when the anti-Mullerian hormone (AMH) level of the subject is greater than or equal to 3.0 ng/ml, AMH is 1, and c is any value selected from −4.793561 to −3.876481, preferably −4.335021.

22. The method of claim 13, wherein a basis for grouping pre-stored in the grouping step is as follows:
when the calculated probability (p) of poor ovarian response of the subject is <10%, and the ovarian youth index score is >90 points, the grouping step determines that the subject has very good ovarian reserve function;
when the calculated probability (p) of poor ovarian response of the subject is ≥10% and <25%, and the ovarian youth index score is >75 to 90 points, the grouping module determines that the subject has good ovarian reserve function;
when the calculated probability (p) of poor ovarian response of the subject is ≥25% and <50%, and the ovarian youth index score is >50 to 75 points, the grouping step determines that the subject has poor ovarian reserve function; and
when the calculated probability (p) of poor ovarian response of the subject is ≥50%, and the ovarian youth index score is ≤50 points, the grouping step determines that the subject has very poor ovarian reserve function.
